SEQUENCE LISTING (4 sequences)

SEQ ID NO: 1 - DNA, 20 bp, Artificial Sequence
  Description of Artificial Sequence Synthetic primer
ggaccaccgcatctctacat

SEQ ID NO: 2 - DNA, 20 bp, Artificial Sequence
  Description of Artificial Sequence Synthetic primer
ctttctccgcagtttcctca

SEQ ID NO: 3 - DNA, 20 bp, Artificial Sequence
  Description of Artificial Sequence Synthetic primer
atccactgccctaccgagaa

SEQ ID NO: 4 - DNA, 23 bp, Artificial Sequence
  Description of Artificial Sequence Synthetic primer
cttggctctctgtctgtccagtt

(19) United States
(12) Patent Application Publication
(10) Pub. No.: US 2010/0203112 A1
(43) Pub. Date: Aug. 12, 2010

(54) COMPOSITION OF CATIONIC PHOSPHOLIPID NANOPARTICLES FOR EFFECTIVE DELIVERY OF NUCLEIC ACIDS

(75) Inventors: Yu-Kyoung Oh, Seoul (KR); Hye-Jeong Shin, Seoul (KR); Min-Sung Suh, Seongnam-si (KR); Ga Yong Shim, Yongin-si (KR)

Correspondence Address: ROTHWELL, FIGG, ERNST & MANBECK, P.C., 1425 K STREET, N.W., SUITE 800, WASHINGTON, DC, 20005, US

(73) Assignee: SNU R&DB FOUNDATION, Seoul (KR)

(21) Appl. No.: 12/593,773

(22) PCT Filed: Mar. 28, 2008
(86) PCT No.: PCT/KR08/01753

§ 371 Date: Apr. 5, 2010

(30) Foreign Application Priority Data
Mar. 29, 2007 (KR) 10-2007-0030730

(51) Int. Cl. A61K 9/127; A61P 35/04; A61K 31/7088; C12N 5/02

(52) U.S. Cl. 424/450; 514/44 R; 514/44 A; 435/375

(57) ABSTRACT

The present invention provides a cationic phospholipid liposome composition comprising 1,2-dioleoyl-sn-glycero-S-ethylphosphocholine (EDOPC), 3β-[N—(N′,N′-dimethylaminoethane)-carbamoyl] cholesterol (DC-cholesterol) and 1,2-diphytanoyl-sn-glycero-3-phosphoethanolamine (DPhPE), a liposome-nucleic acid complex which is capable of forming a complex therewith, and a pharmaceutical composition comprising the same. The cationic phospholipid liposome of the present invention is highly effective for intracellular delivery of nucleic acids and reduction of cytotoxicity, as compared to conventional liposome products. Therefore, the present invention can be useful for gene therapy via intracellular delivery of a desired material to target cells.

### TECHNICAL FIELD

The present invention relates to a cationic phospholipid liposome composition comprising 1,2-dioleoyl-sn-glycero-3-ethylphosphocholine (EDOPC), 3β-[N—(N′,N′-dimethylaminoethane)-carbamoyl]cholesterol (DC-cholesterol) and 1,2-diphytanoyl-sn-glycero-3-phosphoethanolamine (DPhPE), a liposome-nucleic acid complex which is capable of forming a complex therewith, and a pharmaceutical composition comprising the same.

### BACKGROUND ART

Methods of introducing nucleic acids into animal or human subjects are known for a variety of beneficial applications in the gene delivery field. Since some techniques were proposed in the mid 1960's for the treatment of genetically linked diseases via the intracellular insertion of normal gene sequences into genetic disease patients harboring incomplete or defective gene sequences, many attempts have been made to treat a variety of genetic diseases and disorders via the gene treatment. Further, gene therapy has been recently proposed for the treatment of cancer.

Diverse techniques and delivery systems, particularly vector systems have been suggested for intracellular delivery of genes of interest to many kinds of target cells. Techniques adopted for delivery or expression of nucleic acids may be broadly divided into viral vector systems and non-viral vector systems. In particular, cationic liposomes or cationic polymers among the non-viral vector systems have been studied as promising gene delivery systems, because they are positively charged due to structural characteristics and then bind to negatively charged genes to thereby form a complex. When compared with viral gene delivery systems using a viral vector such as Lentivirus, Adenovirus, or the like, gene delivery approaches based on such synthetic delivery vector systems provide various advantages such as easy and convenient preparation of delivery systems, non-limited size of genes to be delivered, low immunological side effects even after repeated administration by viral capsid proteins, no potential risk associated with in vivo safety of viral genes per se, and commercially advantageous low production costs and time.

With regard to cationic polymer delivery systems out of the gene delivery methods using cationic materials, a great deal of research has focused on DEAE dextran, polylysine consisting of a repeated structure of lysine as an amino acid unit, polyethyleneimine consisting of a repeated structure of ethyleneimine, and polyamidoamine (U.S. Pat. No. 6,020,457) as the gene delivery system. However, such cationic polymer-based gene delivery systems were reported to suffer from low efficiency of in vivo intracellular delivery, as compared to viral vector delivery systems where gene transfer is effectively made through cell surface receptors. On the other hand, cationic phospholipid-based gene delivery systems have employed a method of mixing a phospholipid composition with positively charged lipid in a certain ratio to prepare nano-sized particles such as cationic liposomes, mixing the resulting nanoparticles with genes of interest to prepare a positively charged phospholipid-gene complex and then treating cell lines with the complex to thereby enhance the expression of the desired gene (U.S. Pat. No. 5,858,784).

Approaches to increase the gene transfer efficiency using cationic phospholipid liposomes have been reportedly made with a variety of cell lines in the art. For example, it is known that a cationic phospholipid liposome composed of cationic phospholipid 3b-[N—(N′,N′-dimethylaminoethane)-carbamoyl]cholesterol (hereinafter, referred to as “DC-cholesterol”) and cell-fusogenic phospholipid dioleoyl phosphatidylethanolamine (hereinafter, referred to as “DOPE”) may be used as a delivery system for a variety of genes (Aberle A. M. et al., Biochemistry 37, 6533-6540 (1998); Colosimo, A., et al., Biochim. Biophys. Acta 1419, 186, (1999); and Villaret D. et al., Head & Neck 24, 661-669 (2002)). In addition, Lipofectamine™ 2000 (Invitrogen, USA) containing cationic lipid is commercially available for an investigational purpose and has been widely used as a transfection agent in the art. However, these gene transfer systems unfortunately have various shortcomings associated with potential cytotoxicity, significant fluctuations in the gene transfer efficiency depending upon kinds of cell lines and therefore consequent difficulty in versatile applications of the systems to various cells, and poor gene transfer efficiency. Therefore, there is an urgent need in the art for development of a technique that is capable of achieving efficient intracellular delivery of nucleic acids (such as plasmid genes, small interfering RNAs (siRNAs), and the like) with low cytotoxicity, via use of a non-viral delivery system.

### DISCLOSURE OF THE INVENTION

### Technical Problem

Therefore, the present invention has been made in view of the above problems, and it is an object of the present invention to provide a cationic phospholipid liposome composition which is capable of achieving attenuated cytotoxicity and enhancing intracellular delivery of desired materials, for example nucleic acids.

It is another object of the present invention to provide a liposome-nucleic acid complex wherein the aforesaid cationic phospholipid liposome is bound to a nucleic acid.

It is a further object of the present invention to provide a pharmaceutical composition for the treatment of diseases via intracellular delivery of nucleic acids, a use of the same pharmaceutical composition, and a method for the treatment of diseases using the same pharmaceutical composition.

It is a still further object of the present invention to provide a use of a pharmaceutical composition for the preparation of a therapeutic agent to treat tumors or genetic diseases.

It is yet another object of the present invention to provide a method for delivery of nucleic acids to animal cells.

### Technical Solution

In accordance with an aspect of the present invention, the above and other objects can be accomplished by the provision of a cationic phospholipid liposome composition comprising 1,2-dioleoyl-sn-glycero-3-ethylphosphocholine (EDOPC), 3β-[N—(N′,N′-dimethylaminoethane)-carbamoyl]cholesterol (DC-cholesterol) and 1,2-diphytanoyl-sn-glycero-3-phosphoethanolamine (DPhPE).

As used herein, the term “liposome” refers to a closed multilayered structure formed by an outer lipid bilayer enclosing an aqueous inner compartment. In particular, the liposome of the present invention is present in the form of a multilamellar vesicle (MLV), having multiple stacks of lipid bilayers. Further, the liposome of the present invention is composed of a multilayered structure of cationic phospholipids DC-cholesterol and DPhPE, and cell-fusogenic phospholipid EDOPC. In other words, the liposome of the present invention is a form of nanoparticles having cationic properties.

The liposome may be prepared by a conventional method well-known in the art. Typically, the multilamellar vesicles of the present invention may be prepared by a lipid-film hydration technique. Specifically, the aforementioned phospholipid components EDOPC, DC-cholesterol and DPhPE are each dissolved in a suitable organic solvent, for example methanol, ethanol, dimethylsulfoxide, chloroform or a mixture thereof, and the organic solvent is then removed to form a thin film. There is no particular limit to the method of removing the solvent. Therefore, removal of the solvent may be carried out by any conventional method known in the art, including rotary evaporation and nitrogen purging.

Next, an aqueous medium is added to hydrate the resulting thin film to thereby prepare a liposome of the present invention.

Based on the total weight of liposomal lipids, each content of EDOPC, DC-cholesterol, and DPhPE, which are incorporated into the liposome composition of the present invention, is 3 to 45% by weight, preferably 6 to 35% by weight for EDOPC, 3 to 45% by weight, preferably 6 to 35% by weight for DC-cholesterol, and 10 to 94% by weight, preferably 30 to 88% by weight for DPhPE. The objects of the present invention can be most effectively achieved at the above-specified ranges of EDOPC, DC-cholesterol and DPhPE.

As will be illustrated hereinafter, there is no particular limit to a particle diameter of the liposome, as long as liposome-mediated delivery of a desired material to animal cells is not interfered with. According to the present invention, the liposomes may be sized to have substantially homogeneous sizes in a given particle diameter range, typically between about 10 nm to 500 nm, preferably 30 nm to 450 nm. One effective sizing method for the liposomes involves extruding an aqueous suspension of the liposome through a series of polycarbonate membranes having a given uniform membrane pore size in the range of 30 nm to 200 nm, typically 50 nm, 100 nm, or 200 nm. The pore size of the membrane corresponds approximately to an average size of the liposome produced by extrusion through that membrane. Particularly, in order to achieve a homogeneous size of the prepared liposome, the extrusion is carried out many times, for example more than three times, through a membrane filter having the same membrane pore size. Homogenization methods are also useful for down-sizing liposomes to sizes of preferably 300 nm or less (Martin, F. J., in SPECIALIZED DRUG DELIVERY SYSTEMS-MANUFACTURING AND PRODUCTION TECHNOLOGY, (P. Tyle, Ed.) Marcel Dekker, New York, pp. 267-316 (1990)).

The liposome of the present invention as constructed above and having a nano-sized particle diameter may be used for another object of the present invention, i.e., effective delivery of a desired material, preferably nucleic acid, to a variety of animal cells. Due to having reduced cytotoxicity, the liposome of the present invention may also be effectively used in gene therapy which involves intracellular delivery of a desired material, preferably nucleic acid.

In accordance with another aspect of the present invention, there is provided a liposome-nucleic acid complex wherein the aforesaid liposome composition is bound to a nucleic acid of interest.

As used herein, “nucleic acid” is intended to encompass DNAs, RNAs, oligonucleotides, aptamers, double stranded RNAs (ds-RNAs), plasmid DNAs, and small interfering RNAs (siRNAs). Also included in this term are derivatives of these molecules which are substituted by oxygen atoms present in phosphate and ester moieties of the nucleic acid structure, other atoms such as sulfur and fluorine atoms, or alkyl groups such as methyl. Preferred are plasmid DNAs and siRNAs.

In the context of the present invention, the term “binds to” or “bound to” as here used in connection with the liposome-nucleic acid complex refers to the state where one or more polycations and negatively charged nucleic acid molecules are connected to each other via the charge-charge interaction. Nucleic acid strands may form a more compact structure via the formation of a complex with the liposome.

The liposome composition of the present invention has cationic properties, whereas the nucleic acid has anionic properties. Therefore, due to the presence of positive charges of the cationic phospholipid liposome and negative charges of the nucleic acid, the liposome and the nucleic acid may form a liposome-nucleic acid complex via electrostatic bonding, even when they are simply mixed.

A nucleic acid in the liposome-nucleic acid complex binds to the liposome at a concentration of at least 0.5 μg/μmol of liposomal lipid. The nucleic acid may bind to the liposome at a concentration of preferably 0.1 to 100 μg/μmol of liposomal lipid, and more preferably about 0.5 to about 50 μg/μmol of liposomal lipid. In one embodiment of the present invention, about 2 to 20 μg of siRNA or plasmid gene/μmmol of liposomal lipid forms a complex with the liposome. A content of nucleic acid in the complex may vary depending upon how long a solution containing the liposome of the present invention and a solution containing the nucleic acid are mixed and left. Preferably, the amount of nucleic acid may be determined by allowing the mixture to stand at room temperature for about 10 min to 1 hour, more preferably 15 min to 30 min.

The thus-prepared liposome-nucleic acid complex can effectively deliver a desired material, e.g. nucleic acid to the target site, e.g. animal cells in need of introduction of the nucleic acid. Therefore, the liposome-nucleic acid complex of the present invention can provide effective delivery of nucleic acid to the human cervical epithelial carcinoma cell lines SiHa and/or HeLa, the VK2 cell line, the murine hepatoma cell line Hepa1-6 and/or the human hepatoma cell lines Hep3B and/or Huh7. Specifically, the liposome-nucleic acid complex increases the intracellular delivery efficiency of ds-siRNAs and plasmid DNAs by about 15% or higher, as compared to conventional liposomal formulations (see Tables 1 and 2, and FIGS. 1 and 7).

Further, the liposome-nucleic acid complex of the present invention can be safely used due to a significant decrease in cytotoxicity that may occur upon delivery of a desired material, i.e. nucleic acid, to animal target cells, as compared to cationic phospholipid liposomes which are commercially available in the market or are disclosed in the literature (Aberle A. M. et al., Biochemistry 37, 6533-6540 (1998)).

The liposome of the present invention that forms a complex with nucleic acid exhibited a ubiquitous increase of the gene transfer efficiency in remarkably diverse cell lines, as compared to conventional nucleic acid delivery systems. Therefore, the liposome composition of the present invention not only increases intracellular nucleic acid delivery, but also exhibits significantly decreased cytotoxicity thereof for a broader spectrum of cells, when compared with cationic phospholipid liposomes composed of DC-cholesterol and DOPE which have been conventionally used to enhance intracellular delivery of nucleic acids. Therefore, the liposome composition of the present invention may be effectively used for gene therapy using nucleic acids, preferably siRNAs or plasmid genes.

In order to augment in vivo targetability of a liposome-nucleic acid complex into target cells, the liposome-nucleic acid complex composition of the present invention, as will be illustrated hereinafter, may further comprise one or more lipid derivatives (such as galactolipids, mannosylated lipids, folate-lipid conjugates, PEG-lipid conjugates, and biotinylated lipids). A content of the lipid derivative may be in a range of about 0 to 20% by weight, preferably 1 to 15% by weight, and more preferably 2 to 10% by weight, based on the total weight of liposomal lipids.

Examples of the galactolipid may include cerebrosides, cerebroside sulfates, and cerebroside sulfatides; Examples of the folate-lipid conjugate may include 1,2-distearoyl-sn-glycero-3-phosphoethanolamine-N-[folate(polyethylene glycol)-2000], 1,2-dipalmitoyl-sn-glycero-3-phosphoethanolamine-N-[folate(polyethylene glycol)-2000], and 1,2-dioleoyl-sn-glycero-3-phosphoethanolamine-N-[folate(polyethylene glycol)-2000]; Examples of the PEG-lipid conjugate may include 1,2-diacyl-sn-glycero-3-phosphoethanolamine-N-[methoxy(polyethylene glycol)-1000], 1,2-diacyl-sn-glycero-3-phosphoethanolamine-N-[methoxy(polyethylene glycol)-2000], and 1,2-diacyl-sn-glycero-3-phosphoethanolamine-N-[methoxy(polyethylene glycol)-5000]; and Examples of the biotinylated lipid may include 1,2-distearoyl-sn-glycero-3-phosphoethanolamine-N-[methoxy(polyethylene glycol)-2000], 1,2-dipalmitoyl-sn-glycero-3-phosphoethanolamine-N-[methoxy(polyethylene glycol)-2000], and 1,2-dioleoyl-sn-glycero-3-phosphoethanolamine-N-[methoxy(polyethylene glycol)-2000].

In accordance with yet another aspect of the present invention, there is provided a pharmaceutical composition comprising the aforesaid liposome-nucleic acid complex. The pharmaceutical composition may further comprise one or more pharmaceutically acceptable carriers.

As used herein, the term “pharmaceutically acceptable carrier” refers to a medium that is generally acceptable for use in connection with the administration of liposomal bioactive agents and liposomal formulations into animals including humans. Pharmaceutically acceptable carriers are generally formulated according to a number of factors well within the purview of an ordinarily skilled artisan to determine and account for, including without limitation: the particular liposomal bioactive agent to be used, and its concentration, stability and intended bioavailability; the disease, disorder or condition being treated with the liposome-nucleic acid complex; the subject, and its age, size and general condition; and the composition's intended route of administration, e.g., nasal, oral, ophthalmic, topical, transdermal, or intramuscular. Typical pharmaceutically acceptable carriers used in parenteral bioactive agent administration include, for example, D5W, an aqueous solution containing 5% by volume of dextrose and physiological saline. Further, the pharmaceutically acceptable carrier may further include additional ingredients which can enhance the stability of active ingredients, such as preservatives, antioxidants, and the like. Furthermore, the composition and liposome-nucleic acid complex of the present invention may be preferably formulated into a desired dosage form, depending upon diseases to be treated and ingredients, using any appropriate method known in the art, as disclosed in “Remington's Pharmaceutical Sciences,” (latest edition), Mack Publishing Co., Easton, Pa.

The pharmaceutical composition of the present invention may be administered by any conventional route known in the art. The composition is administered in a therapeutically effective amount, depending upon intended therapeutic applications. The therapeutically effective amount of the active drug that is required to treat a certain medical disease or inhibit a further progress thereof may be easily determined by those skilled in the art during pre clinical and clinical trials. For adults, the composition may be administered at a single dose of 0.000001 mg/kg/day to 100 mg/kg/day. As used herein, the term “therapeutically effective amount” refers to an amount of an active ingredient that will elicit the biological or medical response of a tissue, a system, an animal or a human that is being sought by a clinician or a researcher.

Further, the present invention provides a method for the treatment of tumors or genetic diseases, comprising administering the aforesaid pharmaceutical composition to an animal.

Further, the present invention provides a use of the aforesaid pharmaceutical composition for the treatment of tumors or genetic diseases.

Further, the present invention provides a use of the aforesaid pharmaceutical composition for the preparation of a therapeutic agent to treat tumors or genetic diseases.

Further, the present invention provides a method for delivery of nucleic acids to animal cells, comprising (1) preparing a cationic phospholipid liposome of the present invention; (2) binding the resulting cationic phospholipid liposome with a nucleic acid to form a liposome-nucleic acid complex; and (3) contacting animal cells with the resulting liposome-nucleic acid complex.

Steps (1) and (2) are as illustrated hereinbefore. Contacting of Step 3 includes addition of a liposome-nucleic acid complex composition to a culture medium surrounding cells in vitro or in vivo, or administration of a pharmaceutical composition comprised of a liposome-nucleic acid complex and a pharmaceutically acceptable carrier into an animal or animal cells in vitro or in vivo. As used herein, the term “animal” is intended to encompass any mammal including humans.

In vitro or in vivo introduction of exogenous nucleic acids using a delivery system may remedy a diversity of cellular defects arising from underexpression or overexpression of genes, or otherwise may modify cell proteins and expression thereof. Intracellular delivery of nucleic acids by the medium of the liposome of the present invention which forms a complex with nucleic acids of interest may be therapeutically effective for the treatment of animals suffering from disorders or diseases caused by abnormal or substantially no detectable protein expression levels, or inordinately low or high protein expression levels. Examples of disorders and diseases that can be treated by the present invention may include, but are not limited to, a variety of cancers and tumors, asthma, arthritis, immunological diseases, and gene-defect diseases.

Further details concerning genetic engineering technologies in the context of the present invention can be found in the literature: Sambrook, et al. Molecular Cloning, A Laboratory Manual, Cold Spring Harbor Laboratory Press, Cold Spring Harbor, N.Y. (2001); and Frederick M. Ausubel et al., Current Protocols in Molecular Biology, volume 1, 2, 3, John Wiley & Sons, Inc. (1994).

### ADVANTAGEOUS EFFECTS

As will be specifically demonstrated hereinafter, a cationic phospholipid liposome of the present invention, which will form a complex with a nucleic acid of interest and has a novel composition, can provide superior delivery of desired materials, e.g. nucleic acids, to various types of animal cells, as compared to conventional cationic phospholipid liposomes. Further, due to a significant decrease in cytotoxicity, this liposome-nucleic acid complex can be useful for effective intracellular delivery of desired nucleic acids to a subject which is in need of gene therapy or any treatment regimen.

### DESCRIPTION OF THE DRAWINGS

FIG. 1 shows phase-contrast micrographs (A,B) and fluorescence micrographs (C,D) illustrating intracellular delivery of siRNA in SiHa cell lines, conducted using fluorescent marker-labeled siRNA for cationic phospholipid liposome of Comparative Example 1 (A,C) and cationic phospholipid liposome of Example 1 (B,D);

FIG. 2 shows phase-contrast micrographs (A,B) and fluorescence micrographs (C,D) illustrating intracellular delivery of siRNA in HeLa cell lines, conducted using fluorescent marker-labeled siRNA for cationic phospholipid liposome of Comparative Example 1 (A,C) and cationic phospholipid liposome of Example 3 (B,D);

FIG. 3 shows phase-contrast micrographs (A,B) and fluorescence micrographs (C,D) illustrating intracellular delivery of siRNA in Hepa1-6 cell lines, conducted using fluorescent marker-labeled siRNA for cationic phospholipid liposome of Comparative Example 1 (A,C) and cationic phospholipid liposome of Example 2 (B,D);

FIG. 4 shows phase-contrast micrographs (A,B) and fluorescence micrographs (C,D) illustrating intracellular delivery of siRNA in Hep3B cell lines, conducted using fluorescent marker-labeled siRNA for cationic phospholipid liposome of Comparative Example 1 (A,C) and cationic phospholipid liposome of Example 4 (B,D);

FIG. 5 shows phase-contrast micrographs (A,B) and fluorescence micrographs (C,D) illustrating intracellular delivery of siRNA in Huh7 cell lines, conducted using fluorescent marker-labeled siRNA for cationic phospholipid liposome of Comparative Example 1 (A,C) and cationic phospholipid liposome of Example 5 (B,D);

FIG. 6 shows a phase-contrast micrograph (A) illustrating cell morphology of a group treatment of a delivery system, and phase-contrast micrographs (B,C) and fluorescence micrographs (D,E) illustrating intracellular delivery of siRNA in VK2 cell lines, conducted for cationic phospholipid liposome of Comparative Example 2 (B,D) and cationic phospholipid liposome of Example 6 (C,E);

FIG. 7 shows phase-contrast micrographs (A,B,C) and fluorescence micrographs (D,E,F) illustrating the expression of a green fluorescent protein (GFP) after intracellular delivery of a liposome-nucleic acid complex to HeLa cell lines, conducted using pEGFP-N2 plasmid DNA harboring genetic information of GFP for cationic phospholipid liposome of Comparative Example 1 (A,D), cationic phospholipid liposome of Comparative Example 2 (B,E), and cationic'phospholipid liposome of Example 7 (C,F);

FIG. 8 shows phase-contrast micrographs (A,B,C) and fluorescence micrographs (D,E,F) illustrating expression of a green fluorescent protein (GFP) after intracellular delivery of a liposome-nucleic acid complex to Hepa1-6 cell lines, conducted using pEGFP-N2 plasmid DNA harboring genetic information of GFP for cationic phospholipid liposome of Comparative Example 1 (A,D), cationic phospholipid liposome of Comparative Example 2 (B,E), and cationic phospholipid liposome of Example 11 (C,F);

FIG. 9 shows a bar graph illustrating the results of cytotoxicity testing in the VK2 cell line, conducted in order to ascertain the fact that cationic phospholipid liposome compositions of Examples 2, 5 and 9 have lower cytotoxicity, as compared to compositions of Comparative Examples 1 and 2;

FIG. 10 shows graphs illustrating the results of fluorescence-activated cell sorter (FACS) analysis for intracellular delivery efficiency of siRNA in HeLa cell lines, conducted for a control group with no treatment of fluorescent marker-labeled siRNA (A), a group with treatment of fluorescent marker-labeled siRNA using no delivery system (B), and each group with treatment of fluorescent marker-labeled siRNA using a composition of Comparative Example 1 (C), a composition of Example 1 (D), a composition of Example 3 (E), and a composition of Example 8 (F), respectively;

FIG. 11 shows graphs illustrating the results of FACS analysis for intracellular delivery of siRNA in SiHa cell lines, conducted for a control group with no treatment of fluorescent marker-labeled siRNA (A), a group with treatment of fluorescent marker-labeled siRNA using no delivery system (B), and each group with treatment of fluorescent marker-labeled siRNA using a composition of Comparative Example 1 (C), a composition of Example 4 (D), a composition of Example 7 (E), and a composition of Example 11 (F), respectively;

FIG. 12 shows micrographs comparing inhibition of transcript expression of a target gene survivin for evaluation of intracellular delivery efficiency of siRNA to SiHa cells through reverse transcriptase-polymerase chain reaction (RT-PCR), conducted for a non-siRNA treated control group (A), a group treated with siRNA capable of specifically interfering and inhibiting the expression of a target gene survivin using no delivery system (B), a group with delivery of siRNA via formation of a complex with a composition of Comparative Example 1 (C), a group with delivery of siRNA via formation of a complex with a composition of Comparative Example 2 (D), and each group with delivery of siRNA via formation of a complex with a composition of Example 2 (E), a composition of Example 7 (F), and a composition of Example 10 (G), respectively;

FIG. 13 shows micrographs comparing inhibition of transcript expression of a target gene survivin for evaluation of intracellular delivery efficiency of siRNA to HeLa cells through RT-PCR, conducted for a non-siRNA treated control group (A), a group treated with siRNA capable of specifically interfering and inhibiting the expression of a target gene survivin using no delivery system (B), a group with delivery of siRNA via formation of a complex with a composition of Comparative Example 1 (C), a group with delivery of siRNA via formation of a complex with a composition of Comparative Example 2 (D), and each group with delivery of siRNA via formation of a complex with a composition of Example 3 (E), a composition of Example 5 (F), and a composition of Example 12 (G), respectively;

FIG. 14 shows micrographs comparing inhibition of transcript expression of a target gene survivin for evaluation of intracellular delivery efficiency of siRNA to Hepa1-6 cells through RT-PCR, conducted for a non-siRNA treated control group (A), a group treated with siRNA capable of specifically interfering and inhibiting the expression of a target gene survivin using no delivery system (B), a group with delivery of siRNA via formation of a complex with a composition of Comparative Example 1 (C), a group with delivery of siRNA via formation of a complex with a composition of Comparative Example 2 (D), and each group with delivery of siRNA via formation of a complex with a composition of Example 6 (E), a composition of Example 9 (F), and a composition of Example 11 (G), respectively; and

FIG. 15 shows the analysis results of protein expression obtained after a complex of siGL2 (scrambled siRNA) with a composition of Example 3 and a complex of siRFP with a composition of Example 3 were administered to right and left tumor tissues of mice, respectively. A: Expression levels of RFP proteins in mouse tumor tissues, as measured for whole body of mice. B: Expression levels of RFP proteins upon administration of the complex of siGL2 (scrambled siRNA) with a composition of Example 3, as measured for left tumor tissues obtained after dissection of mice. C: Expression levels of RFP proteins upon administration of the complex of siRFP (RFP-specific siRNA) with a composition of Example 3, as measured for right tumor tissues obtained after dissection of mice. D: Quantitative analysis results for mean red fluorescence intensity of RFP in the excised tumor tissues.

### MODE FOR INVENTION

Now, the present invention will be described in more detail with reference to the following Examples. These examples are provided only for illustrating the present invention and should not be construed as limiting the scope and spirit of the present invention.

### Comparative Example 1

### Preparation of Conventional Cationic Liposome

Cationic phospholipid DC-cholesterol (Avanti Polar Lipids Inc., USA) and cell-fusogenic phospholipid DOPE (Avanti Polar Lipids Inc., USA) were each dissolved in 1 mL of chloroform. Then, each of the resulting solutions was taken in a molar ratio of 1:1, mixed in a 10 mL glass septum vial (Pyrex, USA), and then rotary-evaporated at a low speed under a nitrogen atmosphere until chloroform was completely evaporated, thereby preparing a lipid thin film. For preparation of multilamellar vesicles (MLVs), 1 mL of a phosphate-buffered solution (PBS) was added to the above-prepared thin film, and the vial was then sealed, followed by vortexing for 3 min. To obtain a uniform particle size, the film solution was passed three times through a 0.2 μm polycarbonate membrane using an extruder (Northern Lipids Inc., Canada). The resulting cationic phospholipid liposome was stored at 4° C. until use.

### Comparative Example 2

### Conventional Commercially Available Cationic Liposome

Lipofectamine™ 2000 (Invitrogen, USA), which is a conventional cationic liposome formulation commercially available for nucleic acid delivery experiments, was purchased and used according to the manufacturer's instructions.

### Example 1

### Preparation of Cationic Phospholipid Liposome

Cationic phospholipids EDOPC (Avanti Polar Lipids Inc., USA) and DC-cholesterol (Avanti Polar Lipids Inc., USA) and cell-fusogenic phospholipid DPhPE (Avanti Polar Lipids Inc., USA) were each dissolved in 1 mL of chloroform. Then, each of the resulting solutions was taken in amounts of 10% by weight, 10% by weight and 80% by weight, respectively, based on the total weight of EDOPC, DC-cholesterol, and DPhPE, mixed in a 10 mL glass septum vial (Pyrex, USA), and then rotary-evaporated at a low speed under a nitrogen atmosphere until chloroform was completely evaporated, thereby preparing a lipid thin film. For preparation of multilamellar vesicles (MLVs), 1 mL of PBS was added to the above-prepared thin film, and the vial was sealed, followed by vortexing for 3 min. To obtain a uniform particle size, the film solution was passed three times through a 0.2 μm polycarbonate membrane using an extruder (Northern Lipids Inc., Canada). The resulting cationic phospholipid liposome was stored at 4° C. until use.

### Example 2

### Preparation of Cationic Phospholipid Liposome

Based on the total weight of EDOPC, DC-cholesterol, and DPhPE, 15% by weight of EDOPC, 15% by weight of DC-cholesterol and 70% by weight of DPhPE were mixed in a Pyrex glass vial. A cationic phospholipid liposome was then prepared in the same manner as in Example 1.

### Example 3

### Preparation of Cationic Phospholipid Liposome

Based on the total weight of EDOPC, DC-cholesterol, and DPhPE, 20% by weight of EDOPC, 30% by weight of DC-cholesterol and 50% by weight of DPhPE were mixed, in a Pyrex glass vial. A cationic phospholipid liposome was then prepared in the same manner as in Example 1.

### Example 4

### Preparation of Cationic Phospholipid Liposome

Analogously to Example 1, a cationic phospholipid liposome was prepared using 10% by weight of EDOPC, 30% by weight of DC-cholesterol and 60% by weight of DPhPE, based on the total weight of EDOPC, DC-cholesterol, and DPhPE.

### Example 5

### Preparation of Cationic Phospholipid Liposome

Analogously to Example 1, a cationic phospholipid liposome was prepared using 30% by weight of EDOPC, 35% by weight of DC-cholesterol and 35% by weight of DPhPE, based on the total weight of EDOPC, DC-cholesterol, and DPhPE.

### Example 6

### Preparation of Cationic Phospholipid Liposome

Analogously to Example 1, a cationic phospholipid liposome was prepared using 35% by weight of EDOPC, 15% by weight of DC-cholesterol and 50% by weight of DPhPE, based on the total weight of EDOPC, DC-cholesterol, and DPhPE.

### Example 7

### Preparation of Cationic Phospholipid Liposome (Containing Galactolipid)

Based on the total weight of EDOPC, DC-cholesterol, DphPE, and galactolipid, 15% by weight of EDOPC, 10% by weight of DC-cholesterol and 70% by weight of DPhPE, and 5% by weight of galactolipid (cerebroside, Avanti Polar Lipids Inc., USA) were mixed in a 10 mL glass septum vial (Pyrex, USA). A cationic phospholipid liposome was then prepared in the same manner as in Example 1.

### Example 8

### Preparation of Cationic Phospholipid Liposome (Containing Galactolipid)

Based on the total weight of EDOPC, DC-cholesterol, DphPE, and galactolipid, 18% by weight of EDOPC, 7% by weight of DC-cholesterol and 65% by weight of DPhPE, and 10% by weight of galactolipid (cerebroside, Avanti Polar Lipids Inc., USA) were mixed in a 10 mL glass septum vial (Pyrex, USA). A cationic phospholipid liposome was then prepared in the same manner as in Example 1.

### Example 9

### Preparation of Cationic Phospholipid Liposome (Containing Folate-Lipid Conjugate)

Based on the total weight of EDOPC, DC-cholesterol, DphPE, and folate-lipid conjugate, 10% by weight of EDOPC, 22% by weight of DC-cholesterol and 60% by weight of DPhPE, and 8% by weight of a folate-lipid conjugate (1,2-distearoyl-sn-glycero-3-phosphoethanolamine-N-[folate(polyethylene glycol)-2000], Avanti Polar Lipids Inc., USA) were mixed in a 10 mL glass septum vial (Pyrex, USA). A cationic phospholipid liposome was then prepared in the same manner as in Example 1.

### Example 10

### Preparation of Cationic Phospholipid Liposome (Containing PEG-Lipid Conjugate)

Based on the total weight of EDOPC, DC-cholesterol, DphPE, and PEG-lipid conjugate, 30% by weight of EDOPC, 20% by weight of DC-cholesterol and 45% by weight of DPhPE, and 5% by weight of a PEG-lipid conjugate (1,2-diacyl-sn-glycero-3-phosphoethanolamine-N-[methoxy(polyethylene glycol)-1000], Avanti Polar Lipids Inc., USA) were mixed in a 10 mL glass septum vial (Pyrex, USA). A cationic phospholipid liposome was then prepared in the same manner as in Example 1.

### Example 11

### Preparation of Cationic Phospholipid Liposome (Containing PEG-Lipid Conjugate)

Based on the total weight of EDOPC, DC-cholesterol, DphPE, and PEG-lipid conjugate, 27% by weight of EDOPC, 35% by weight of DC-cholesterol and 35% by weight of DPhPE, and 3% by weight of a PEG-lipid conjugate (1,2-diacyl-sn-glycero-3-phosphoethanolamine-N-[methoxy(polyethylene glycol)-1000], Avanti Polar Lipids Inc., USA) were mixed in a 10 mL glass septum vial (Pyrex, USA). A cationic phospholipid liposome was then prepared in the same manner as in Example 1.

### Example 12

### Preparation of Cationic Phospholipid Liposome (Containing Biotinylated Lipid)

Based on the total weight of EDOPC, DC-cholesterol, DphPE, and biotinylated lipid, 28% by weight of EDOPC, 30% by weight of DC-cholesterol and 40% by weight of DPhPE, and 2% by weight of biotinylated lipid (1,2-distearoyl-sn-glycero-3-phosphoethanolamine-N-methoxy(polyethylene glycol)-2000] (Ammonium Salt)], Avanti Polar Lipids Inc., USA) were mixed in a 10 mL glass septum vial (Pyrex, USA). A cationic phospholipid liposome was then prepared in the same manner as in Example 1.

Materials and Methods for Examples 13 Through 25 and Experimental Example 1

SiHa, HeLa, Hepa1-6, VK2, Hep3B and Huh7 cell lines were purchased from American Type Culture Collection (ATCC, USA). The SiHa, Hepa1-6, Hep3B, and Huh7 cell lines were cultured in Dulbecco's Modified Eagle's Medium (DMEM, Gibco, USA) containing 10% w/v fetal bovine serum (FBS, HyClone Laboratories Inc., USA) and 100 unit/mL of penicillin or 100 μg/mL of streptomycin. The HeLa cell line was cultured in RPMI 1640 (Gibco, USA) supplemented with 10% FBS, penicillin and streptomycin. The VK2 cell line was cultured in Keratinocyte-SFM (Gibco, USA) supplemented with 0.1 ng/mL of a recombinant human epidermal growth factor (rhEGF, Gibco, USA), 0.05 mg/mL of bovine pituitary extract (BPE, Gibco, USA) and 100 unit/mL of penicillin or 100 μg/mL streptomycin.

### Example 13

### Delivery of siRNA into SiHa Cell Line

On the day prior to the experiment, SiHa cells were seeded on 24-well plates at a density of 8×10⁴ cells/well. When cells of each plate were grown to 60% to 70% confluence, culture media of the plates were replaced with 500 μl/well of fresh media. 50 μl of serum-free medium was added to Eppendorf tubes to which 2 μl of Block-iT™ Fluorescent Oligo (20 μmol, Invitrogen, USA) as fluorescent marker-labeled siRNA, and 10 μl of cationic phospholipid liposomes prepared in Comparative Example 1 and Example 1 were then added. These materials were slowly pipetted, mixed and allowed to stand at room temperature for 20 min, thus resulting in formation of a complex. The thus-prepared complex was added to the well plate, followed by cell culture in a CO₂ incubator at 37° C. for 24 hours. The SiHa cell-cultured medium was replaced with 500 μl/well of a fresh medium, and the gene transfer efficiency was examined under a fluorescence microscope.

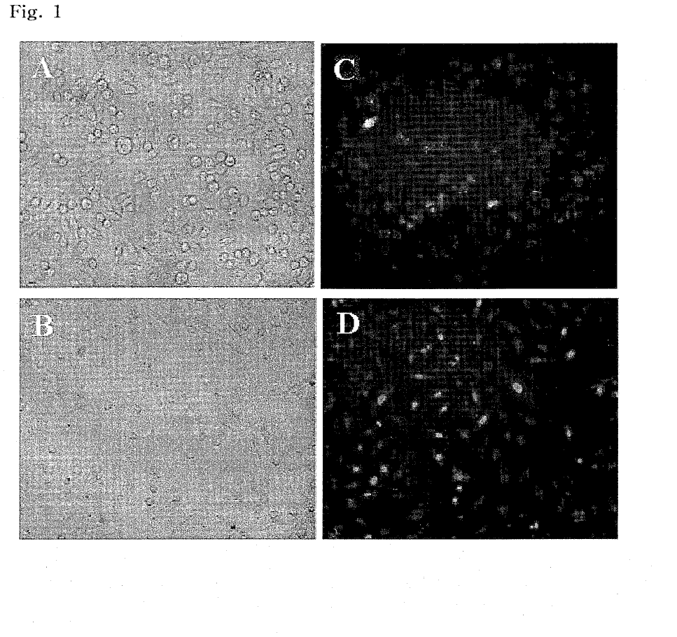

FIG. 1 shows phase-contrast and fluorescence microscopic observations illustrating siRNA delivery efficiency of cationic phospholipid liposomes prepared in Comparative Example 1 (A,C) and Example 1 (B,D). A: Phase-contrast microscopic image when treated with the liposome composition of Comparative Example 1. B: Phase-contrast microscopic image when treated with the liposome composition of Example 1. C: Fluorescence microscopic image illustrating intracellular delivery of fluorescent marker-labeled siRNA when treated with a complex of nucleic acid and liposome composition of Comparative Example 1. D: Fluorescence microscopic image illustrating intracellular delivery fluorescent marker-labeled siRNA when treated with a complex of nucleic acid and liposome composition of Example 1. From the results of FIG. 1, it can be seen that the cationic phospholipid liposome of Example 1 exhibits increased siRNA delivery efficiency into SiHa cells, as compared to the liposome of Comparative Example 1 with a known composition.

### Example 14

### Delivery of siRNA into HeLa Cell Line

On the day prior to the experiment, HeLa cells were seeded on 24-well plates at a density of 8×10⁴ cells/well. When cells of each plate were grown to 60% to 70% confluence, culture media of the plates were replaced with 500 μl/well of fresh media. Analogously to Example 13, a complex was prepared in which each liposome of Comparative Example 1 and Example 3 was conjugated with Block-iT™ Fluorescent Oligo as fluorescent marker-labeled siRNA. The thus-prepared complex was added to the well plate, followed by cell culture in a CO₂ incubator at 37° C. for 24 hours. The HeLa cell-cultured medium was replaced with 500 μl/well of a fresh medium, and the gene transfer efficiency was examined under a fluorescence microscope.

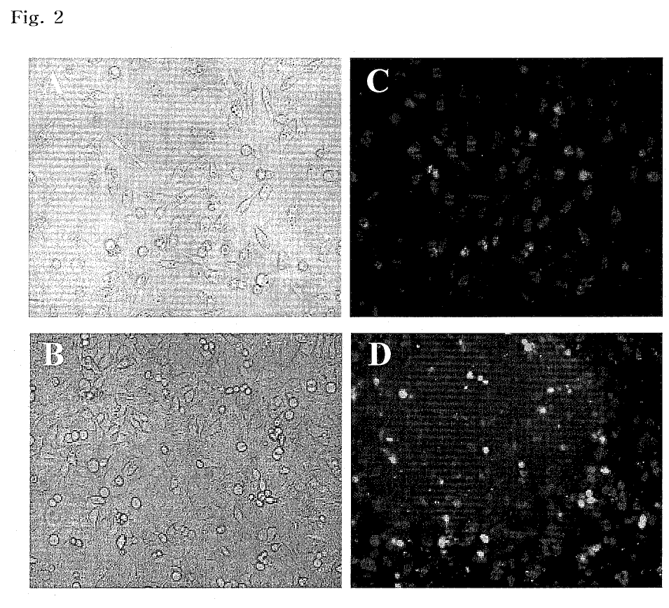

FIG. 2 shows phase-contrast and fluorescence microscopic observations illustrating siRNA delivery efficiency of cationic phospholipid liposomes prepared in Comparative Example 1 (A,C) and Example 3 (B,D). A: microscopic image when treated with the liposome composition of Comparative Example 1. B: Phase-contrast microscopic image when treated with the liposome composition of Example 3. C: Fluorescence microscopic image illustrating intracellular delivery of fluorescent marker-labeled siRNA when treated with the liposome composition of Comparative Example 1. D: Fluorescence microscopic image illustrating intracellular delivery of fluorescent marker-labeled siRNA when treated with the liposome composition of Example 3. From the results of FIG. 2, it can be seen that the cationic phospholipid liposome of Example 3 exhibits increased siRNA delivery efficiency into HeLa cells, as compared to the liposome of Comparative Example 1 with a known composition.

### Example 15

### Delivery of siRNA into Hepa1-6 Cell Line

On the day prior to the experiment, Hepa1-6 cells were seeded on 24-well plates at a density of 8×10⁴ cells/well. When cells of each plate were grown to 60% to 70% confluence, culture media of the plates were replaced with 500 μg/well of fresh media. Analogously to Example 13, each complex of liposomes of Comparative Example 1 and Example 2 with Block-iT™ Fluorescent Oligo was prepared. The thus-prepared complex was added to the well plate, followed by cell culture in a CO₂ incubator at 37° C. for 24 hours. The Hepa1-6 cell-cultured medium was replaced with 500 μl/well of a fresh medium, and the RNA delivery efficiency was examined under a fluorescence microscope.

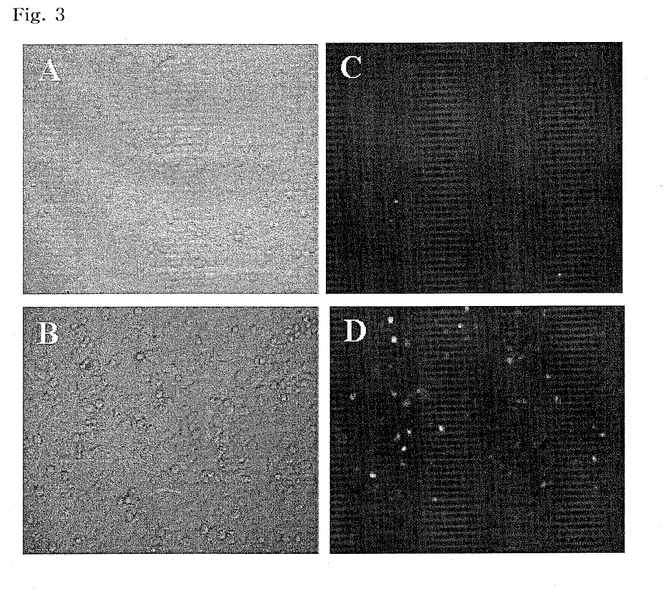

FIG. 3 shows phase-contrast and fluorescence microscopic observations illustrating RNA delivery efficiency of cationic phospholipid liposomes prepared in Comparative Example 1 (A,C) and Example 2 (B,D). A: Phase-contrast microscopic image when treated with the liposome composition of Comparative Example 1. B: Phase-contrast microscopic image when treated with the liposome composition of Example 2. C: Fluorescence microscopic image illustrating intracellular delivery of fluorescent marker-labeled siRNA when treated with the liposome composition of Comparative Example 1. D: Fluorescence microscopic image illustrating intracellular delivery of fluorescent marker-labeled siRNA when treated with the liposome composition of Example 2. From the results of FIG. 3, it can be seen that the cationic phospholipid liposome of Example 2 exhibits increased siRNA delivery efficiency into Hepa1-6 cells, as compared to the liposome of Comparative Example 1.

### Example 16

### Delivery of siRNA into Hep3B Cell Line

On the day prior to the experiment, Hep3B cells were seeded on 24-well plates at a density of 8×10⁴ cells/well. When cells of each plate were grown to 60% to 70% confluence, culture media of the plates were replaced with 500 μl/well of fresh media. Analogously to Example 13, each complex of liposomes of Comparative Example 1 and Example 4 with Block-iT™ Fluorescent Oligo was prepared. The thus-prepared complex was added to the well plate, followed by cell culture in a CO₂ incubator at 37° C. for 24 hours. The Hep3B cell cultured medium was replaced with 500 μl/well of a fresh medium, and the gene transfer efficiency was examined under a fluorescence microscope.

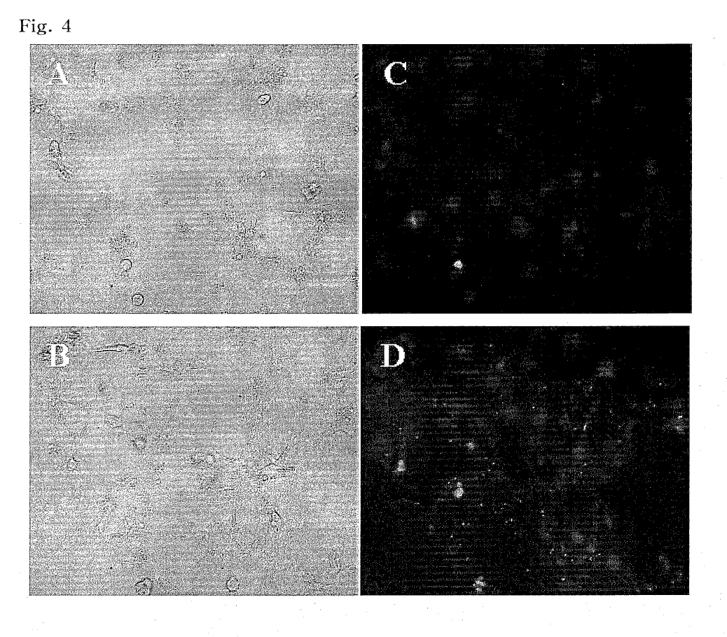

FIG. 4 shows phase-contrast and fluorescence microscopic observations illustrating gene transfer efficiency of cationic phospholipid liposomes prepared in Comparative Example 1 (A,C) and Example 4 (B,D). A: Phase-contrast microscopic image when treated with the liposome composition of Comparative Example 1. B: Phase-contrast microscopic image when treated with the liposome composition of Example 4. C: Fluorescence microscopic image illustrating intracellular delivery of fluorescent marker-labeled siRNA when treated with the liposome composition of Comparative Example 1. D: Fluorescence microscopic image illustrating intracellular delivery of fluorescent marker-labeled siRNA when treated with the liposome composition of Example 4. From the results of FIG. 4, it can be seen that the cationic phospholipid liposome of Example 4 exhibits increased siRNA delivery efficiency into Hep3B cells, as compared to the liposome of Comparative Example 1.

### Example 17

### Delivery of siRNA into Huh7 Cell Line

On the day prior to the experiment, Huh7 cells were seeded on 24-well plates at a density of 8×10⁴ cells/well. When cells of each plate were grown to 60% to 70% confluence, culture media of the plates were replaced with 500 μl/well of fresh media. Analogously to Example 13, each complex of liposome compositions of Comparative Example 1 and Example 5 with fluorescence-labeled ds-siRNA was prepared. The thus-prepared complex was added to the well plate, followed by cell culture in a CO₂ incubator at 37° C. for 24 hours. The Huh7 cell-cultured medium was replaced with 500 μl/well of a fresh medium, and the gene transfer efficiency was examined under a fluorescence microscope.

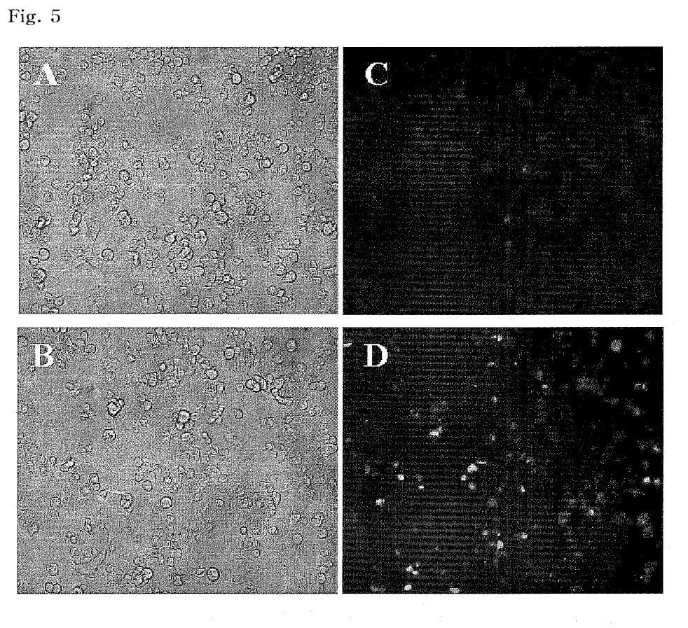

FIG. 5 shows phase-contrast and fluorescence microscopic observations illustrating siRNA delivery efficiency of cationic phospholipid liposomes prepared in Comparative Example 1 (A,C) and Example 5 (B,D). A: Phase-contrast microscopic image when treated with the liposome composition of Comparative Example 1. B: Phase-contrast microscopic image when treated with the liposome composition of Example 5. C: Fluorescence microscopic image illustrating intracellular delivery of fluorescent marker-labeled siRNA when treated with the liposome composition of Comparative Example 1. D: Fluorescence microscopic image illustrating intracellular delivery of fluorescent marker-labeled siRNA when treated with the liposome composition of Example 5. From the results of FIG. 5, it can be seen that the cationic phospholipid liposome of Example 5 exhibits increased siRNA delivery efficiency into Huh7 cells, as compared to the liposome of Comparative Example 1.

### Example 18

### Delivery of siRNA into VK2 Cell Line

On the day prior to the experiment, VK2 cells were seeded on 24-well plates at a density of 8×10⁴ cells/well. When cells of each plate were grown to 60% to 70% confluence, culture media of the plates were replaced with 500 μl/well of fresh media. Analogously to Example 13, each complex of liposome compositions of Comparative Example 2 and Example 6 with fluorescence-labeled ds-siRNA was prepared. The thus-prepared complex was added to the well plate, followed by cell culture in a CO₂ incubator at 37° C. for 24 hours. The VK2 cell cultured medium was replaced with 500 μl/well of a fresh medium, and the siRNA delivery efficiency was examined under a fluorescence microscope.

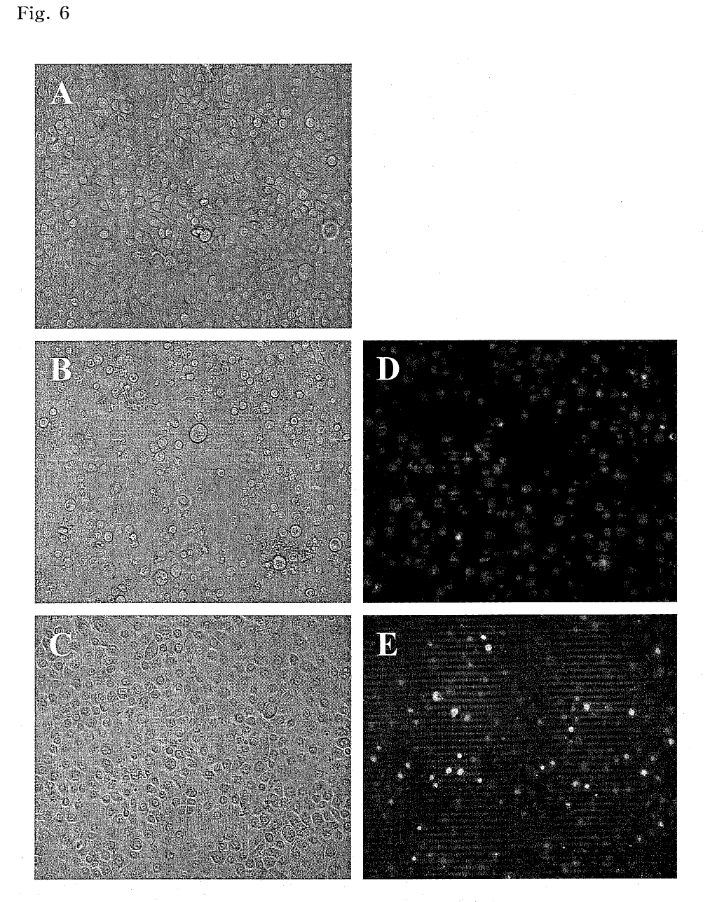

FIG. 6 shows phase-contrast micrograph (A) illustrating cell morphology of a group with no treatment of a delivery system, and phase-contrast and fluorescence microscopic observations illustrating siRNA delivery efficiency of commercially available cationic phospholipid liposome of Comparative Example 2 (B,D) and cationic phospholipid liposome of Example 6 (C,E). From the results of FIG. 6, it can be seen that the cationic phospholipid liposome of Example 6 exhibits increased siRNA delivery efficiency into VK2 cells, as compared to the liposome of Comparative Example 2. Further, as shown in FIG. 6B in terms of cell morphology observed under a phase-contrast microscope, most of cells exhibited cell shrinkage when treated with commercially available Lipofectamine™ 2000 (Comparative Example 2), thus representing poor health state of the cells. On the other hand, when the cells were treated with the liposome composition of Example 6, the cells, as shown in FIG. 6C, exhibited morphology similar to that of a non-treated control group, thus representing a significant decrease in cytotoxicity.

### Example 19

### Delivery of Plasmid DNA into HeLa Cell Line

On the day prior to the experiment, HeLa cells were seeded on 24-well plates at a density of 8×10⁴ cells/well. When cells of each plate were grown to 60% to 70% confluence, culture media of the plates were replaced with 500 μl/well of fresh media. 50 μl of serum-free medium was added to Eppendorf tubes to which 0.8 μg of the plasmid pEGFP-N2 (Clontech) capable of expressing a green fluorescent protein (GFP) (1.35 μg/μl) and 10 μl of cationic phospholipid liposomes prepared in Comparative Examples 1 and 2 and Example 7 were then added. These materials were slowly pipetted, mixed and allowed to stand at room temperature for 20 min, thus resulting in formation of a complex. The thus-prepared complex was added to the well plate, followed by cell culture for 36 hours. The HeLa cell-cultured medium was replaced with 500 μl/well of a fresh medium, and the plasmid gene transfer efficiency was examined under a fluorescence microscope.

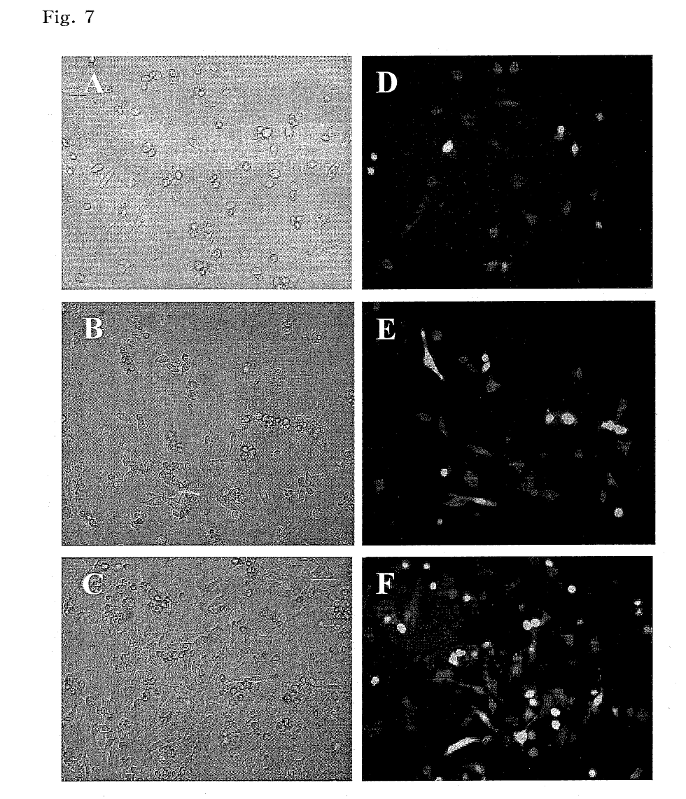

FIG. 7 shows phase-contrast and fluorescence microscopic observations illustrating the plasmid gene transfer efficiency of cationic phospholipid liposomes prepared in Comparative Example 1 (A,D), Comparative Example 2 (B,E), and Example 7 (C,F). A: Phase-contrast microscopic image when treated with the liposome composition of Comparative Example 1. B: Phase-contrast microscopic image when treated with the liposome composition of Comparative Example 2. C: Phase-contrast microscopic image when treated with the liposome composition of Example 7. D: Fluorescence microscopic image illustrating the expression of a green fluorescent protein (GFP) after intracellular delivery of a plasmid having genetic information of GFP to HeLa cells, when treated with the liposome composition of Comparative Example 1. E: Fluorescence microscopic image when treated with the liposome composition of Comparative Example 2. F: Fluorescence microscopic image when treated with the liposome composition of Comparative Example 7. From the results of FIG. 7, it can be seen that the cationic phospholipid liposome of Example 7 exhibits increased plasmid gene transfer efficiency into HeLa cells, as compared to the liposomes of Comparative Examples 1 and 2.

### Example 20

### Delivery of Plasmid DNA into Hepa1-6 Cell Line

On the day prior to the experiment, Hepa1-6 cells were seeded on 24-well plates at a density of 8×10⁴ cells/well. When cells of each plate were grown to 60% to 70% confluence, culture media of the plates were replaced with 500 μl/well of fresh media. Each of the pEGF-N2/cationic liposome complexes, which were prepared analogously to Example 19 from cationic phospholipid liposomes of Comparative Examples 1 and 2 and Example 11, was added to well plates, followed by cell culture for 36 hours. The Hepa1-6 cell-cultured medium was replaced with 500 μl/well of a fresh medium, and the plasmid gene transfer efficiency was examined under a fluorescence microscope.

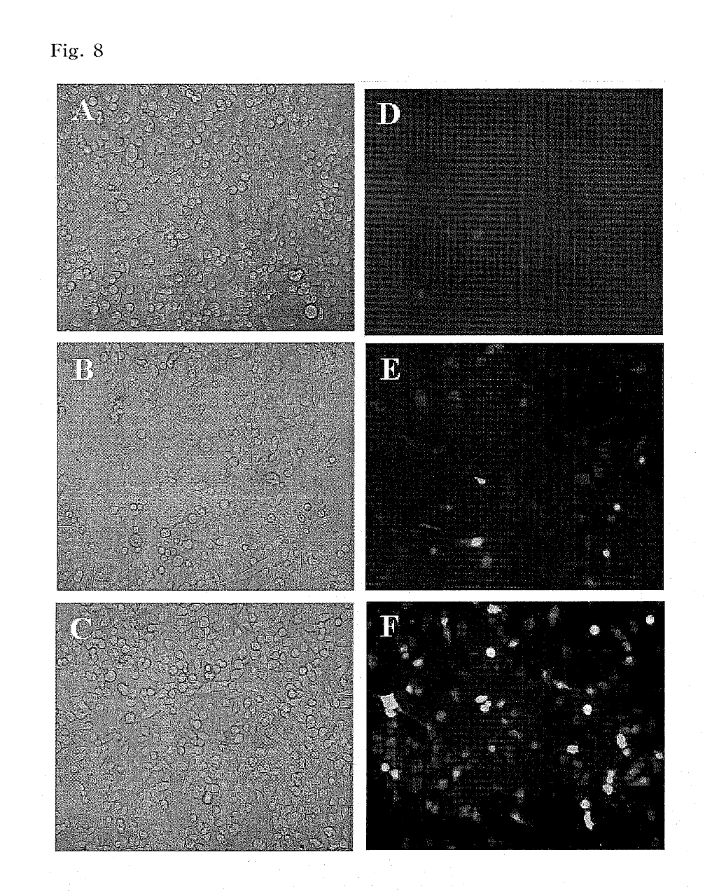

FIG. 8 shows phase-contrast and fluorescence microscopic observations illustrating the plasmid gene transfer efficiency of cationic phospholipid liposomes prepared in Comparative Example 1 (A,D), Comparative Example 2 (B,E), and Example 11 (C,F). A: Phase-contrast microscopic image when treated with the liposome composition of Comparative Example 1. B: Phase-contrast microscopic image when treated with the liposome composition of Comparative Example 2. C: Phase-contrast microscopic image when treated with the liposome composition of Example 11. D: Fluorescence microscopic image illustrating the expression of a green fluorescent protein (GFP) after intracellular delivery of a plasmid having genetic information of GFP to Hepa1-6 cells, when treated with the liposome composition of Comparative Example 1. E: Fluorescence microscopic image when treated with the liposome composition of Comparative Example 2. F: Fluorescence microscopic image when treated with the liposome composition of Comparative Example 11. From the results of FIG. 8, it can be seen that the cationic phospholipid liposome of Example 11 exhibits increased plasmid gene transfer efficiency into Hepa1-6 cells, as compared to the liposomes of Comparative Examples 1 and 2.

### Experimental Example 1

### Cytotoxicity Test of Liposome-Nucleic Acid Complexes in VK2 Cell Line

The cytotoxicity of a complex composed of a cationic phospholipid liposome used to improve intracellular delivery efficiency of siRNA or plasmid was assayed according to the following experiment. VK2-cells were treated with each complex of siRNA with cationic phospholipid liposomes of Comparative Examples 1 and 2 and Examples 2, 5 and 9 and with the siRNA gene per se, and the cytotoxicity was evaluated for individual cell groups. The toxicity assay was carried out using the MTT (3-(4,5-dimethylthiazol-2-yl)-2,5-diphenyl tetrazolium bromide) colorimetric assay. 1×10⁴ cells/well of VK2 cells were seeded onto a 96-well plate and cultured for 12 hours. Thereafter, the cells were treated with a complex composition composed of 10 μmol of siRNA and 1 μl of the cationic phospholipid liposome of Comparative Example 2, complex compositions composed of 40 pmol of siRNA and 4 μl of cationic phospholipid liposomes of Comparative Example 1 and Examples 2, 5 and 9, and 40 pmol of the siRNA gene per se. 24 hours after treatment of the cells with individual test complexes, an MTT solution was added to make 10% of the medium, followed by cell culture for another 4 hours. The supernatant was discarded and a 0.04 N isopropanol hydrochloride solution was added to the medium. Then, absorbance values were measured at 570 nm using an ELISA reader (Tecan, Switzerland). Cells with no treatment of a cationic phospholipid liposome/gene complex were used as a control group.

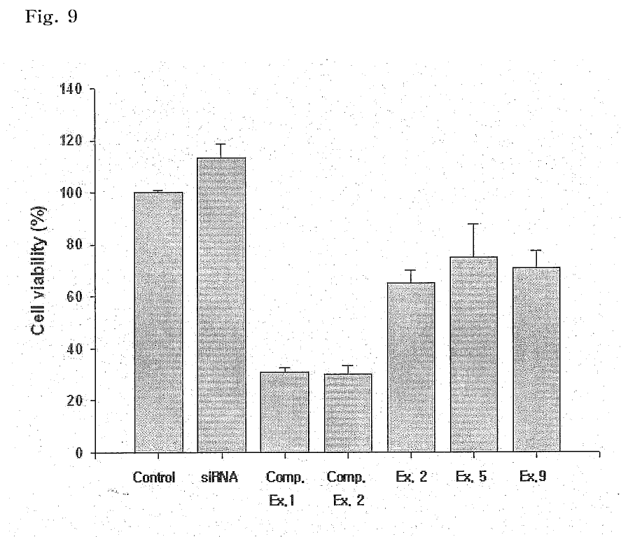

FIG. 9 shows the results of cytotoxicity test in the VK2 cell line, conducted for complexes of siRNA with 4 μl of cationic phospholipid liposome compositions of Comparative Examples 1 and 2 and Examples 2, 5 and 9, and a complex of siRNA with 1 μl of the cationic phospholipid liposome composition of Comparative Example 2. As can be seen from FIG. 9, the complexes of siRNA with cationic phospholipid liposomes of Comparative Examples 1 and 2 exhibited significantly higher cytotoxicity, as compared to the control group. On the other hand, the complexes of siRNA with 4 μl of cationic liposomes of Examples 2, 5 and 9 exhibited relatively low cytotoxicity. Therefore, it can be seen that the cationic phospholipid liposomes of Examples 2, 5 and 9 provide reduced cytotoxicity in the VK2 cell line, as compared to the liposomes of Comparative Examples 1 and 2.

### Example 21

### Delivery of siRNA into HeLa Cell Line—Fluorescence Activated Cell Sorting (FACS) Analysis

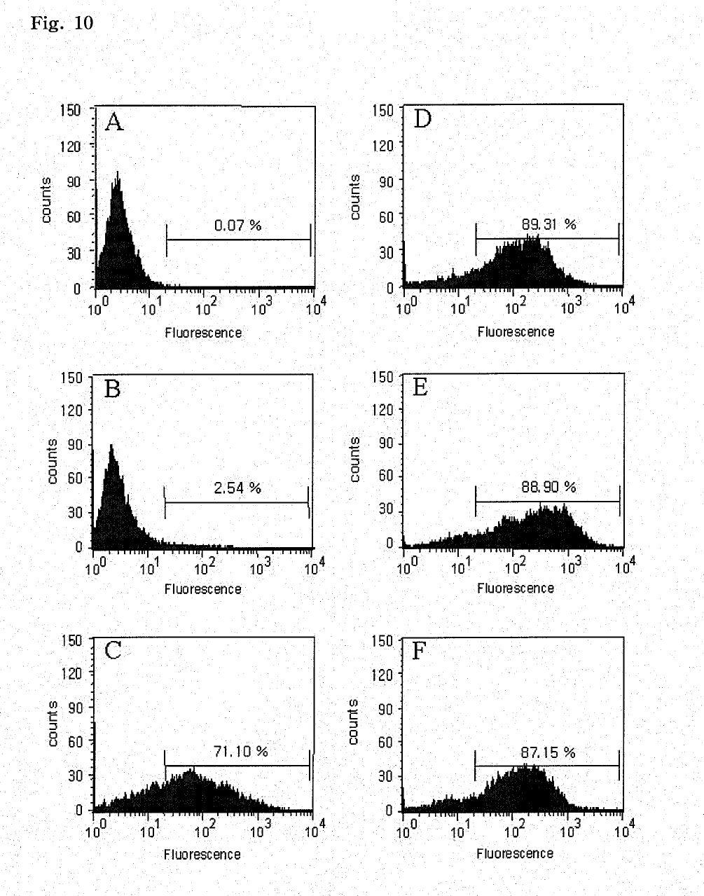

On the day prior to the experiment, HeLa cells were seeded on 6-well plates at a density of 3×10⁵ cells/well. When cells of each plate were grown to 60% to 70% confluence, culture media of the plates were replaced with 800 μl/well of fresh media. 100 μl of serum-free medium was added to Eppendorf tubes to which 2 μl of Block-iT™ Fluorescent Oligo (20 μmol, Invitrogen, USA) as fluorescence-labeled siRNA, and 10 μl of cationic phospholipid liposomes prepared in Comparative Example 1 and Examples 1, 3 and 8 were then added. These materials were slowly pipetted, mixed and allowed to stand at room temperature for 20 min, thus resulting in formation of a complex. The thus-prepared complex was added to the well plate, followed by cell culture in a CO₂ incubator at 37° C. for 24 hours. The cultured cells were collected and washed two times with PBS. The cells with incorporation of fluorescence-labeled siRNA were analyzed for intracellular delivery efficiency of siRNA by means of shift of fluorescence intensity peaks using a BD FACS Calibur flow cytometry system (BD Biosciences, USA). The results obtained are shown in FIG. 10. Further, the FACS analysis results for intracellular delivery efficiency of siRNA by individual experimental groups are quantitatively given in Table 1 below. The control group (A) was the non-siRNA-treated group, whereas the siRNA-alone treated group (B) was the siRNA-treated group without use of a delivery system. The control group (A) and the siRNA-alone treated group (B) exhibited substantially no peak shift due to no intracellular delivery of siRNA, whereas the group (C) treated with the liposome of Comparative Example 1 having a known composition exhibited 71.1% delivery of siRNA. On the other hand, the groups treated with the liposomes of Example 1 (D), Example 3 (E), and Example 8 (F) of the present invention exhibited 89.31%, 88.90%, and 87.15%, respectively, thus representing increased intracellular delivery efficiency, as compared to the group (C) treated with the liposome of Comparative Example 1. From these results, it can be seen that the cationic phospholipid liposomes prepared in Examples of the present invention have increased delivery efficiency of siRNA in HeLa cells, as compared to the liposome prepared in Comparative Example 1.

TABLE 1
siRNA delivery efficiency of cationic phospholipid liposomes
in HeLa cells

| A | B | C | D | E | F |
|---|---|---|---|---|---|
| Control | siRNA-alone treated | Comp. Ex. 1 | Ex. 1 | Ex. 3 | Ex. 8 |
| | group | | | | |
| 0.07% | 2.54% | 71.10% | 89.31% | 88.90% | 87.15% |

### Example 22

### Delivery of siRNA into SiHa Cell Line—FACS Analysis

On the day prior to the experiment, SiHa cells were seeded on 6-well plates at a density of 3×10⁵ cells/well. When cells of each plate were grown to 60% to 70% confluence, culture media of the plates were replaced with 800 μl/well of fresh media. 100 μl of serum-free medium was added to Eppendorf tubes to which 2 μl of Block-iT™ Fluorescent Oligo (20 μmol, Invitrogen, USA) and 10 μl of cationic phospholipid liposomes prepared in Comparative Example 1 and Examples 4, 7 and 11 were then added. These materials were slowly pipetted, mixed and allowed to stand at room temperature for 20 min, thus resulting in formation of a complex. The thus-prepared complex was added to the well plate, followed by cell culture in a CO₂ incubator at 37° C. for 24 hours. The cultured cells were collected and washed two times with PBS. The Block-iT-incorporated cells were analyzed for intracellular delivery efficiency of siRNA by means of shift of fluorescence intensity peaks using a BD FACS Calibur flow cytometry system.

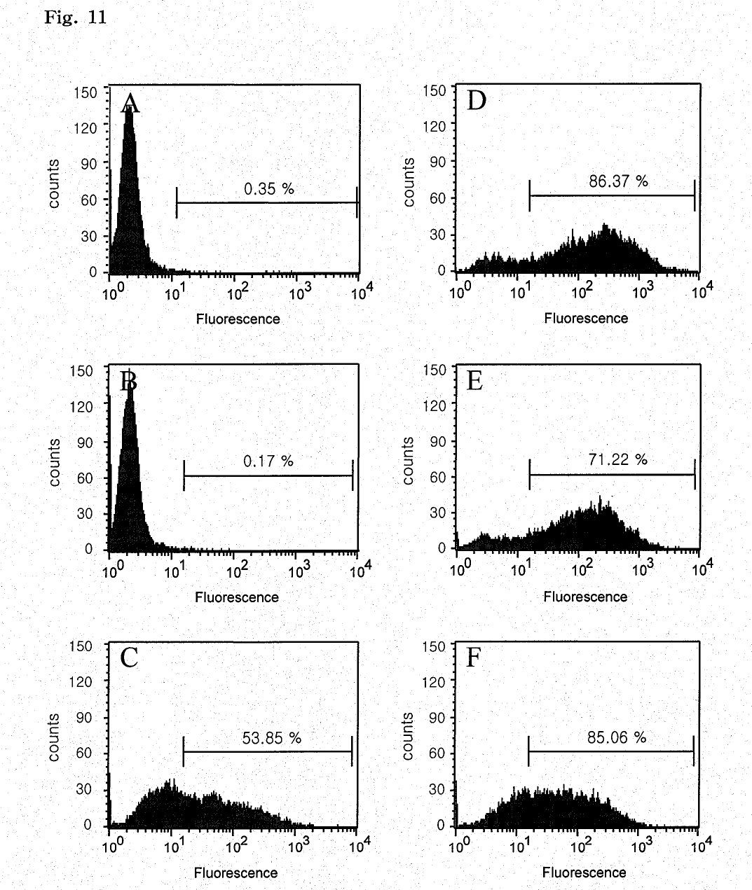

The results obtained are shown in FIG. 11. Further, the FACS analysis results for intracellular delivery efficiency of siRNA by individual experimental groups are quantitatively given in Table 2 below. The control group (A) was the non-siRNA-treated group, whereas the siRNA-alone treated group (B) was the siRNA-treated group without use of a delivery system. The control group (A) and the siRNA-alone treated group (B) exhibited substantially no peak shift due to no intracellular delivery of siRNA, whereas the group (C) treated with the liposome of Comparative Example 1 having a known composition exhibited 53.85% delivery of siRNA. On the other hand, the groups treated with the liposomes of Example 4 (D), Example 7 (E), and Example 11 (F) of the present invention all exhibited more than 70% delivery of siRNA, thus representing increased intracellular delivery efficiency of siRNA, as compared to the group (C) treated with the liposome of Comparative Example 1. From these results, it can be seen that the cationic phospholipid liposomes prepared in Examples of the present invention have increased delivery efficiency of siRNA in SiHa cells, as compared to the liposome prepared in Comparative Example 1.

TABLE 2
siRNA delivery efficiency of cationic phospholipid liposomes
in SiHa cells

| A | B | C | D | E | F |
|---|---|---|---|---|---|
| Control | siRNA- | Comp. Ex. 1 | Ex. 4 | Ex. 7 | Ex. 11 |
| | alone treated group | | | | |
| 0.35% | 0.17% | 53.85% | 86.37% | 71.22% | 85.06% |

### Example 23

### Delivery of siRNA into SiHa Cell Line—Reverse Transcriptase-Polymerase Chain Reaction (RT-PCR) Analysis

On the day prior to the experiment, SiHa cells were seeded on 24-well plates at a density of 8×10⁴ cells/well. When cells of each plate were grown to 60% to 70% confluence, culture media of the plates were replaced with 250 μl/well of fresh media. 25 μl of serum-free medium was added to Eppendorf tubes to which 30 pmol of siRNA and 10 μl of cationic phospholipid liposomes prepared in Comparative Examples 1 and 2, and Examples 2, 7 and 10 were then added. siRNA to induce the inhibition of expression of the survivin gene (GenBank accession number: NM_{—}001168) was constructed using siGENOME SMARTpool (Dharmacon, Lafayette, Colo., USA). A final concentration of siRNA in the media was adjusted to 100 nM. These materials were slowly pipetted, mixed and allowed to stand at room temperature for 20 min, thus resulting in formation of a complex. The thus-prepared complex was added to the well plate, followed by cell culture in a CO₂ incubator at 37° C. for 24 hours. After 24 hours, total RNA was isolated from cultured cells using Trizol reagent (Invitrogen, Carlsbad, Calif., USA) and then reverse-transcribed into cDNA using AccuPower RT PreMix (Bioneer, Daejeon, Korea). The survivin-specific primer had a sequence of 5′-GGACCACCG CATCTCTACAT-3′ (forward) and 5′-CTTTCTCCGCAGTTTCCTCA-3′ (reverse), and the size of the polymerase chain reaction (PCR) product was 347 by in length. Expression of the survivin gene was assayed by determining quantitative changes of the gene expression through normalization of a band density of the survivin-specific PCR product against a band density appeared by amplification of the GAPDH (glyceraldehyde-3-phosphate dehydrogenase) gene.

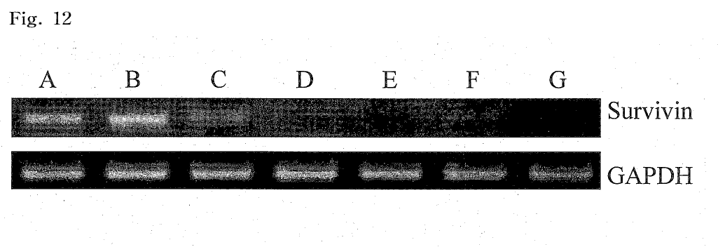

FIG. 12 shows micrographs comparing transcript expression of a target gene survivin in SiHa cells, when cells were treated with individual compositions. A: Control group, B: siRNA-alone treated group, C: Group treated with the composition of Comparative Example 1, D: Group treated with the composition of Comparative Example 2, E: Group treated with the composition of Example 2, F: Group treated with the composition of Example 7, and G: Group treated with the composition of Example 10. The control group (A) and the siRNA-alone treated group (B) exhibited no changes in expression of the survivin gene due to no intracellular delivery of survivin-specific siRNA, whereas the group (C) treated with the liposome of Comparative Example 1 exhibited a slight decrease in the expression of the survivin gene, as compared to the groups treated with the liposomes of Examples 2, 7 and 10. The liposomes of Examples 2, 7 and 10 exhibited efficient attenuation of survivin gene expression even while having lower cytotoxicity as compared to the commercially available liposome product of Comparative Example 2. From these results, it can be seen that the cationic phospholipid liposomes prepared in Examples 2, 7 and 10 can provide selective suppression of target gene expression via the intracellular delivery of siRNA into SiHa cells.

### Example 24

### Delivery of siRNA into Hela Cell Line—RT-PCR Analysis

On the day prior to the experiment, HeLa cells were seeded on 24-well plates at a density of 8×10⁴ cells/well. When cells of each plate were grown to 60% to 70% confluence, culture media of the plates were replaced with 250 μl/well of fresh media. Under the same method and conditions as in Example 23, the subsequent experiment was carried out using the liposomes prepared in Comparative Examples 1 and 2, and Examples 3, 5 and 12.

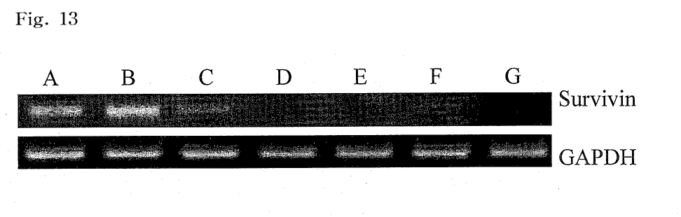

FIG. 13 shows micrographs comparing transcript expression of a target gene survivin in HeLa cells, when cells were treated with individual compositions. A: Control group, B: siRNA-alone treated group, C: Group treated with the composition of Comparative Example 1, D: Group treated with the composition of Comparative Example 2, E: Group treated with the composition of Example 3, F: Group treated with the composition of Example 5, and G: Group treated with the composition of Example 12. The control group (A) and the siRNA-alone treated group (B) exhibited no changes in expression of the survivin gene due to no intracellular delivery of siRNA, whereas the group (C) treated with the liposome of Comparative Example 1 exhibited a slight decrease in the expression of the survivin gene, as compared to the groups treated with the liposomes of Examples 3, 5 and 12. The liposomes of Examples 3, 5 and 12 exhibited efficient attenuation of survivin gene expression even while having lower cytotoxicity as compared to the commercially available liposome product of Comparative Example 2. From these results, it can be seen that the cationic phospholipid liposomes prepared in Examples 3, 5 and 12 can provide selective suppression of target gene expression via the intracellular delivery of siRNA into HeLa cells.

### Example 25

### Delivery of siRNA into Hepa1-6 Cell Line—RT-PCR Analysis

On the day prior to the experiment, Hepa1-6 (mouse hepatoma cell line) cells were seeded on 24-well plates at a density of 8×10⁴ cells/well. When cells of each plate were grown to 60% to 70% confluence, culture media of the plates were replaced with 250 μl/well of fresh media. 25 μl of serum-free medium was added to Eppendorf tubes to which 30 pmol of siRNA and 10 μl of cationic phospholipid liposomes prepared in Comparative Examples 1 and 2 and Examples 6, 9 and 11 were then added. The siRNA sequence to induce the inhibition of expression of the survivin gene (GenBank accession number: NM_{—}009689) was custom-made by Samchully Pharm. Co., Ltd. (Seoul, Korea). A final concentration of siRNA in the media was adjusted to 100 nM. These materials were slowly pipetted, mixed and allowed to stand at room temperature for 20 min, thus resulting in formation of a complex. The thus-prepared complex was added to cells which were then cultured in a CO₂ incubator at 37° C. for 24 hours. After 24 hours, total RNA was isolated from cultured cells using Trizol reagent (Invitrogen, Carlsbad, Calif., USA) and then reverse-transcribed into cDNA using AccuPower RT PreMix (Bioneer, Daejeon, Korea). The survivin-specific primer had a sequence of 5′-ATCCACTGCCCTACCGAGAA-3′ (forward) and 5′-CTTGGCTCTCTGTCTGTCCAGTT-3′ (reverse), and the size of the polymerase chain reaction (PCR) product was 200 by in length. Expression of the survivin gene was assayed by determining quantitative changes of the gene expression through normalization of a band density of the survivin-specific PCR product against a band density appeared by amplification of the GAPDH (glyceraldehyde-3-phosphate dehydrogenase) gene.

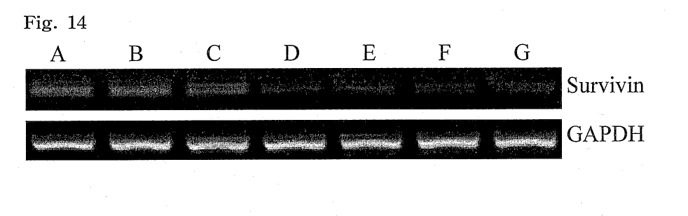

FIG. 14 shows micrographs comparing transcript expression of a target gene survivin in Hepa1-6 cells, when cells were treated with individual compositions. A: Control group, B: siRNA-alone treated group, C: Group treated with the composition of Comparative Example 1, D: Group treated with the composition of Comparative Example 2, E: Group treated with the composition of Example 6, F: Group treated with the composition of Example 9, and G: Group treated with the composition of Example 11. The control group (A) and the siRNA-alone treated group (B) exhibited no changes in expression of the survivin gene due to no intracellular delivery of siRNA, whereas the group (C) treated with the liposome of Comparative Example 1 exhibited a slight decrease in the expression of the survivin gene, as compared to the groups treated with the liposomes of Examples 6, 9 and 11. The liposomes of Examples 6, 9 and 11 exhibited efficient attenuation of survivin gene expression even having lower cytotoxicity as compared to the commercially available liposome product of Comparative Example 2. From these results, it can be seen that the cationic phospholipid liposomes prepared in Examples 6, 9 and 11 can provide selective suppression of target gene expression via the intracellular delivery of siRNA to Hepa1-6 cells.

### Example 26

### Delivery of siRNA into Mouse Tumor Model

A local tumor model was established by subcutaneous injection of 1×10⁶ of the mouse malignant melanoma cell line B16F10 (B16F10-RFP) constructed to express a red fluorescent protein (RFP) into right and left parts of mice. Experimental animals were 5-week old female nu/nu-balb/c mice. siRNA that selectively inhibits the expression of RFP was custom-made by Samchully Pharm. Co., Ltd. (Seoul, Korea). 1 nmol of siRFP, which is siRNA for inhibition of RFP expression, and 100 μl of a delivery system prepared in Example 3 were mixed to prepare a complex. As a negative control group, siGL2 (scrambled siRNA) and 100 μl of a delivery system of Example 3 were mixed to prepare a complex. When a diameter of tumor tissues induced in mice reached to a size of 6 to 7 mm, the complex of scrambled siRNA (siGL2) with the delivery system of Example 3 was directly administered via intratumoral injection to left tumor lesions once a day for 3 days, whereas the complex of RFP-specific siRNA (siRFP) with the delivery system of Example 3 was administered to right tumor lesions.

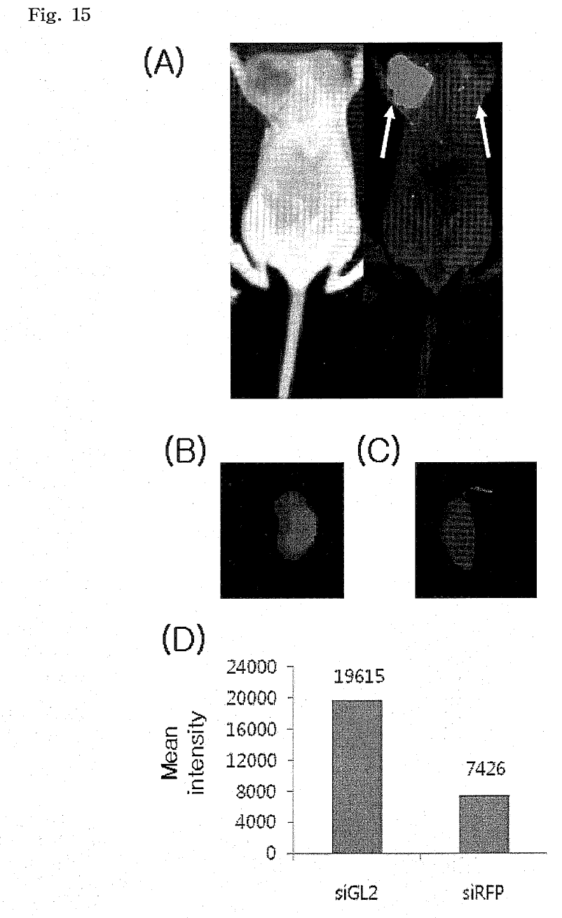

On Day 4 from after the first administration of siRNA, the expression of RFP was confirmed by an in vivo molecular imaging technique. Both right and left tumor tissues were excised and the RFP expression was then assayed. Molecular imaging of tumor lesions in animals was carried out using an Image station 4000MM (KODAK, USA). Quantitative counting of fluorescence in the tissues was made using Kodak molecular imaging software ver.4.0. FIG. 15 shows the analysis results of protein expression obtained after a complex of siGL2 with the composition of Example 3 and a complex of siRFP with the composition of Example 3 were administered to right and left tumor tissues of mice, respectively. A: Expression level of RFP protein in mouse tumor tissues, as measured for whole body of mice. B: Expression level of RFP protein upon administration of the complex of siGL2 (scrambled siRNA) with the composition of Example 3, as measured for left tumor tissues obtained after dissection of mice. C: Expression level of RFP protein upon administration of the complex of siRFP (RFP-specific siRNA) with the composition of Example 3, as measured for right tumor tissues obtained after dissection of mice. D: Quantitative analysis results for mean red fluorescence intensity of RFP in the excised tumor tissues. As shown in FIG. 15A, the right tumor tissues with administration of the siRFP/Example 3 complex exhibited a significant decrease in the expression of RFP, as compared to the left tumor lesions with administration of the siGL2/Example 3 complex. Further, the expression level of RFP in the excised tumor tissues of FIGS. 15B and 15C was significantly decreased in tumor lesions with administration of siRFP and delivery system of Example 3 (FIG. 15C), as compared to tumor lesions with administration of siGL2 and delivery system of Example 3 (FIG. 15B). Upon quantitative comparison of mean red fluorescence intensity between two groups in FIG. 15D, a 2.5-fold decrease in the expression of RFP was observed in tumor lesions treated with the siRFP/Example 3 complex, as compared to tumor lesions treated with the siGL2/Example 3 complex. From these results, it can be seen that the cationic phospholipid liposome prepared in Example 3 can provide selective suppression of target gene expression via the effective delivery of siRNA into tumor cells of the animal model.

### INDUSTRIAL APPLICABILITY

As apparent from the above description, the present invention provides a cationic phospholipid liposome composition comprising 1,2-dioleoyl-sn-glycero-3-ethylphosphocholine (EDOPC), 3β-[N—(N′,N′-dimethylaminoethane)-carbamoyl]cholesterol (DC-cholesterol) and 1,2-diphytanoyl-sn-glycero-3-phosphoethanolamine (DPhPE), liposome-nucleic acid complex which is capable of forming a complex therewith, and a pharmaceutical composition comprising the same. The cationic phospholipid liposome of the present invention is highly effective for intracellular delivery of nucleic acids and reduction of cytotoxicity, as compared to conventional liposome products. Therefore, the present invention can be useful for gene therapy via intracellular delivery of a desired material to target cells.

1. A cationic phospholipid liposome composition comprising 1,2-dioleoyl-sn-glycero-3-ethylphosphocholine (EDOPC), 3β-[N—(N′,N′-dimethylaminoethane)-carbamoyl]cholesterol (DC-cholesterol) and 1,2-diphytanoyl-sn-glycero-3-phosphoethanolamine (DPhPE).

2. The composition of claim 1, wherein the liposome composition comprises 3 to 45% by weight of EDOPC, 3 to 45% by weight of DC-cholesterol and 10 to 94% by weight of DPhPE, based on the total weight of liposomal lipids.

3. The composition of claim 1, wherein the cationic phospholipid liposome has a particle diameter of 30 nm to 450 nm.

4. A liposome-nucleic acid complex wherein a cationic phospholipid liposome composition of claim 1 is bound to a nucleic acid.

5. The complex of claim 4, wherein the nucleic acid is at least one selected from the group consisting of DNA, RNA, oligonucleotide, aptamer, ds-RNA, plasmid DNA, and siRNA.

6. The complex of claim 4, wherein the complex further comprises at least one lipid derivative selected from the group consisting of galactolipid, mannosylated lipid, folate-lipid conjugate, PEG-lipid conjugate, and biotinylated lipid.

7. The complex of claim 6, wherein a content of the lipid derivative is in the range of 1 to 15% by weight, based on the total weight of liposomal lipids.

8. A pharmaceutical composition comprising a liposome-nucleic acid complex of claim 4, for the treatment of tumors or genetic diseases via the intracellular delivery of nucleic acids.

9. The composition of claim 8, wherein the nucleic acid is at least one selected from the group consisting of DNA, RNA, oligonucleotide, aptamer, ds-RNA, plasmid DNA, and siRNA.

10. A method for the treatment of tumors or genetic diseases, comprising administering a pharmaceutical composition of claim 8 to an animal.

11. A use of a pharmaceutical composition of claim 8 for the treatment of tumors or genetic diseases.

12. A use of a pharmaceutical composition of claim 8 for the preparation of a therapeutic agent to treat tumors or genetic diseases.

13. A method for delivery of nucleic acids to animal cells, comprising:
1) preparing a cationic phospholipid liposome composition of claim 1;
2) binding the cationic phospholipid liposome composition with a nucleic acid to form a liposome-nucleic acid complex; and
3) contacting animal cells with the liposome-nucleic acid complex.

14. The method of claim 13, wherein the nucleic acid in Step 2 is at least one selected from the group consisting of DNA, RNA, oligonucleotide, aptamer, ds-RNA, plasmid DNA, and siRNA.
